SEQUENCE LISTING (67 sequences)

SEQ ID NO: 1 - DNA, 12550 bp, Homo sapiens
gatcagataattgtttagttatgccttcaaaaatgtctgagccaggaacagtggataaatgagcttgtgaatcctcgaaaatttgctctttaagttttgaaatatccaaggttaagttatcatcccaggcttttaaatgtcttgagacattttcccagctatgttgatatttattataagcataaggcattatgcaataatcagaagtattccaatcactctgtaattgcatacggtgttccaaattcatatctcccagccagattacactttggcggagatcattaatttgattagctaatttttgatcaatttgagcctgagaattccaaagtctggaggagtttttctgccatgcttcaattcaacatattgaacggtttgaacagaattgtggatggcaactccagcagttgctgctgttgcagtaaccgcaattagtcctgcaatgatggcaataagagtaaaaataaatctctttgttcttttgcggatacctttaagaacttcattgactatgtgaatagagggggaagactcccatggacgatgtaaagaaactggtatccataccccctccctagcccttaccaagagagtacttgtagtgggattaaaagtagcatcaatgcacgtgaacagcttacagttatcacattctatagtttgtgtattgggaatgataattatatttccaaccaacagcatgtaagggggtttgacatagctcctgatgggtatcacccgttcagatatgagggtgatgttgaatgtgggtgttttggtattagtgtgaaggagttgataggtagtgttccatatccttattcctgtcatagctgcagctaattttcatagttgaggatgttctggggtaacaaagggatgaatcatttttggtctaggaggagtaatgcctgcatccatccatttaaataggtaaggggacacccattgtctcagcctgtatgactgccatccatcctctacataatctaacaaataattaaactctgagcatgaggtatttttgctggagcaatcttgccaataatgcccttttggagcccaatcaataacaatacctgcggctagactttggagcacaacggccttaggagcattacaattattccataaaattgaagtcactataaaaggtccctttgtaggtttctttaaccaaaagagaggctgggcacagttgctcacacttgtaaacccagcactttgggagaccgaggcgggcagatcacgaggtcaggagttcaaaaccagcttggccaacatggtgaaacccctgcctcagcttcctgagtgctgggattataggcatgtgccaccacgcgcagctaattcttgtattttttcagtagagacgaggtttcatcatgttgcccaggctggtctcgagtacctgacctcaacctgaggtgatccaaccacctcagcctcccaaagtgctgggattacaagcatgagtcaccgcacccgggccccagtcactttagaatagcatgttgctttgtattcggagggtctctctgcaaatagcccatcaacactgagcgtgcctggaaagacctggttttcaaataactggcttcgtctgtgtaaaacgagtcttgttgtatgcattaaaaattatcttggctgggcgtggtggctcacgcctttaatcccagcattttgggaggctcgttctgttgccagggaggctgcaaaaggaggatgcggctatttctgcttccatcatggaaggctgggattccacaggcacagaaaggaaggagggagagatgggaatgtgactgtcctccagacacagcctctggcaggatcggatgtgggagagtgagggtcccaccccagctggggtctacccaggtccatgtcttggacatgttgagagtttttctggaaggcagggatacagtgtggtccaaaaacacacaaatgcccctactggcccaggggttgtcacaatagactggaagggtgacacatcccaggcgcttgccacccatcacacgcacctcctacccactggcatccttccaccccaggcacacacaaagcctcagtccagagatcaactctggactcagctctgaatttgcatatcctgtgtgtagattcattcttcataacctctgcccagcctagcttgtgtatcatttttttttctctattaggggaggagcccgtcctggcactcccattggcctgtagattcacctcccctgggcagggccccaggacccaggataatatctgtgcctcctgcccagaaccctccaagcagacacaatggtaagaatggtgcctgtcctgctgtctctgctgctgcttctgggtcctgctgtcccccaggagaaccaagatggtgagtggggaaagcaagggatgggtgctggagaggactggaaggaggtgaggaacaggacatgtggctgggagacaggctggatgcagctgggataccctggcatacggcaggaatgggtgcccaaggctgtcaactccctcagctcacacacttccaggagcattcagggagcctctgcgctggcccgaaataagaccttcaggaatctgaatctaaaacccctagtttacagtgaaaacaaagactccaaagaccaagcgacctgcttggggtagacagtcaggacggagtaggaaccatatgcctggagctgcttctgctcctgttccttccctccttccgatggctgggtacacctgcctgacgctgatgaaaagagagagcagccccaaggggaaagtgggaaggcaggttggctggagggatggtgctagaaggaaacccgtgcccaaatcccacactcagacaccactgcagtgggtctggaaggcgagtggctggaagagaagagagtgggagctccgggagatcaagagtcactcctaggataagggaaggaggctgtttgtggcatgagaatgtgcaggataaagacatggaagcgaatggcttctcagttgtgtgagtttaaaattcatgacatttacaaattgtcagaaaaggtgttatatgtttgttatataacaatcactttggaatgttaatctgattctgtgccaaaatctgaattactcagggttctccagagaaacagaactaataggtggtacacatatacatatatatgtacgtacacatacatacatacactgtatacacatggatacacacacacataggaagagatttacatatatgtatacaaaagagagagagagtagagatttattttaagaaattgactcacactattgggaggagtaacaagtcctaaatcttcagagccggccagcaggctggagacccagggaagagttgatgtcttagtcttgattccaagggcagactgtaggcagaattctttcctctttaggggacatctgaggctttttctcttaaggccttcaactgattggatgaagcccaccactatggagagtaatccactttactcaaggtctactgatttttttgtaaattaaaaaaaaaactgtgggtgcatagtatgtgtatatatttatggggtacatgagaggttttgattcaggcatgcaatgtgaaataatcacatcatcaaaaatgaggtatccatcccttcaagcttttatcgtttgtgttacagacaatccaattatacttttttggttattttagtttttaaaagtatttgattatttatttatttatttatttttgagacagagtctcactctgtcacccaggcaggagtgcagtggcatgatctcggctcactgcaacctccgcctcccaggttcaagcaattttcctgcctcagtctcctgagtagctaggactacaggcacctgccaccacacctggctaatttttttgtatttttagtagagacgggtttcatcatgttggccaggctagtcttgatatcctgacctcgtgatctgcccgccttggtctcccaaagtgccgggattacaggtgtcagcaactgcgcctggcctctcttttggttatttaaaagtgtacaattaaattatgattattattattatttttgagatggattcttgttctgtcacccaggctggagtgcagtggcgtgatcttggcttactgcaaacctccgcctgttgggttcaagcaattatcttgcctcgggtgtacactgccacacacggctaacttatgtatttttaatagagatagggtttcaccatgttggctagactggtcttgacctcttgacctcaagtgatccactcacttcagcctcccagagtgctggaattacaggcacgagccaccacacctggccccagttaaattattattgactatagtcaccctgttgtgctatcaaatagtaggtcttattcattcttctttttttttttttttttgtgacagagttgcccaggctggaatgcagtggtgcaatcttggctcactgcaacctctgcctcccgggcttaagcgattctcctgcctcagccttctgagtcgctgggactacaggtgtgtgccaccacgcccggctaatttatgtatttttagtagagatggggtttcaccatgttggccaggctggtttcgaactcctgacctcaagtgacccacctgcctcagcttcccaaagtgttggaattacaggcatgagccaccacacctggccccagttaaattattattcactggagtcactttgttgtgctatcaaatagttttctaactattttttttgtacccattaaccaccctcccaatttccccccaaccctgccactacccttcccagcctttggtaaccatccttctactctctatgtccatgaattcaattgtagggtctactgatttaaaggctaatcacatttagacactcaggagcaagaataattttagtaattgaactaggattctgccatatgacctccaacatcattagcacctgtgtaaattgtatcataaaataattatggaactattatggaaatgtccctctctcccagatcccaccttgtaccaaaatgcaaggtacaaccccgggaattctgagctccatcctagtcttaccctgtgctaattcagtctgggtcatttcttgaattttctggtaaattctcctttctaccctttctaactatatgtatttgtcaggttaagctagaagtgttaattttttttttttttgagatggagccttgctttgtcacctaggctgaagtgcagtggcatgatctcagctcactgcaagctccgcctcccgggttcatgccattctcctgcctcagcctcctgagtagctgggactacaggcacccgccaccatgcttggctaattttgtgaattcttagtagagacggggtttcaccatgttagccaggatggtctcgatctcctgacctcgtgatccacccgcctcggccccctaaagtgctgggattacaggcgtgagccactgagcccggacgaaatgttaatttgttttttttgagacggagtctcactctgtcatccaagctggagtgcagtggcatgatcttggcttgttgcaacctctgcctctctggttcaagtgattttcctgcctcagcctccagcatgactgggattacaggcccgcaccaccatgcccagctaatttttgtattttttaatagagatggggtttcaccatgttggccaggctggtcttcaactcctgatctcaagtaatctgcctgccttggcctcccaaagtcctgggattacaggcatgagccacggagcccagcctagaaatgttaatttctaacgcatgtcagattccatgcacactgggcaaggttccattcctccatggggtgactcagggatccaggccaattgcatattgagactctttcatattatcctgtggccttcaaagtcgtcacctctagggatgagaaacaaaagggacaagccagctggtagggtcttggacaagaagaaagacatcacttctgctcacattctcttttgacaaaactcagtcacatggtcccaatatatcttcgaggtggctgagtaatgttatcttcctatgtgtcaagcagaggaaataatgtagtgaagacacaggatggtctctgaaatatcatctcaggcatgaaagtagagcatattcacttgagtgagcctccagtggtgtgaagttgatggcaggagaaagagctggggaagaaaaggccagtggcaggtctcccctcctagccctatgcagccccacagtgggacccttgcatggacctcaaccatcagaatcttttcttttgcaggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacatcgtggagtattacaacgacagtaacggtcagtgaataacagaccacaggggtggaaggtctaacccaagaggcagcccccccagtgtgagtggcaagggatcagcaggatggaaatagtcccaatcccaggggaagaacaggagacacagcagaaacacagacatgtccacatcccacccaccccacagcacaggtgctccccgcttccccatcaattgccccatcctcatcccaggcctcaggtcacacaggaagtgatggcagagtcacttcctatccaggcacctatgacctctcacctccacaccccacccatcggaggctgatacccccgtgagaaggcatcagactcacccctgtccagggaggttgcctggagagtgagccactctcaaagtcactcagacctgggctcacctggtggttctgccagtcctagctgttgacagtgaaacgttcccaaaatatctggttgaaatctgcaaacattggagcactgagacctacctccaaacaagtctgtaatatttaactatgtctgttctatgaaggatgtcacagtctgtcctgatctcccttgcagctccatcacctagcacagggtacagccaatattggctcaattgaaatttgtggaatccacagagaaaagcacccggcacacaccgtagcccatgctgggggctcaggaagtgctggattcaaaactgtgggctgttagagttccttggagccctaaagttcctccttaccatacgatgcagacccaggaagggccacctgcgctatggtcagaggagctggtggcagagcccgtgcagagatggtccctgtgcccccggcccagtgctctttctcctaaaccacactgccagccccaaggcagccaacctcaggtctggtgaactgctggtgttaaattatcatagagtgggtgtcaaaagatgggctactaagtacaaaaatgcccaaggtgctacatgggatctgaagattttcaaaaggaggcaagaaagagataggcagatgtttcaaggatgtggggtgggggaggtcttggtaaggaaaatggcccaggctgtgtgtcagcaataggagaggagggggcacaggtgatcagaaaagacactgggggaagcattgatggacaggaatagaaatggcaaagtggataattaagaggaaggaggatgaggagatgaacacagggtattagaaaataatagaaggcagggcttggtggctcactcttgtaatcccagcactttgggaggctgaggcaggcagatcacctaaggtcaggagttcgagaccagcccggccaacatggtgaaaccctgtctctactaataatacaaaaatagcctggcatggtggcacacgtctgtggtcccagctactcaggaggctgaggcaggagaattgcttgaacccaggaggcagaggttacagtgagccaaaatcctaccattgcactacagcctgagtgacaagagtgaaacgttgtctaaaaacaaaaaacaaaaaaaggaaataatagtagctgacatttactgagcacttactttgtgccaggcccatctatgagcatatataatgctcagaatagccccctaaaacagtgctcttggcattgccatttcagaggtgaggaaatagaggcacagggagttgagtggctccagttcaggcaacacaccaggtgggggtggggggctggggagagacctgggacgtgagcccagacagcttgagagctttcagagtctatgccaacagcaccaaccagtgctgggtaaacacctgcttttatcatcagaacaaagaggctgtgtcccctgccctatgaggtccatttctgagagttgtggctaatgggcaagaaggttggggctttagagatttgggataaagatatcaaacaccagaaaagtagaaagaagtgatcagattagggttacttaggtgatgatatgaactcttcctagaactgagagaaaaagagagccttcctttactcatatgaaatcacaaataatttctatccaatttggaagtacactttggtgtagttgtgacagcttcctcaggactcagcataaattcaaacaaataattgtccttagaagagatgctatagaagagatagaaatatattcatattctgtagcttttttttttttgagatggatttttgctcttgtcacccaagctggagtgcagtgatgcaatctcagctcactgcaaactttgcctcctgggttcaagggattctcctgcctcagcctcccgataactgggactacaggctacaggcatgtgtcactactcctggttaatttttttttttttttttaagactgagtcttgctctgtctttcaggctgatgtacaatggctccatctcggctcactacaacttctgtcccccaggttcaagcgattctcctgcctcagcctcatgagtagctgggattacaggcatgtgccagcacacccagcaaatttttgtatttttagtagagatgaggtcttaccatgttggccaggctggtctcaaactcctgacctcaggtgatcctttggcctcagcctccctaactgctgggattacaggcatgagccactgcgtccagcctaattttatatttttggtagagatggggtttcaccatattggccaggctggtctcgaactcatgacctaaggtgatccatcctcctcagcctctcaaagtgctgggattacaagtgtgagccactgggcctggtgctttttttttttttttttttttttttttttttttttgagatagggtctcactctgtcacccaggctgaaatgcagtagtgtgattttggctcattgcagccttgacttcccaggctgaagtgatcctcccacctcagcctcctgagtagctggggctacaggcatgcaccaccatgctgcgctaatttttatattttttgtagtggtgggattttgccatatcaccctggctggtctggaacccctgggctcaagcgatccactcgcttcagcttctcaaagtgctgggattacaggcatgagccacagcgcccaggctgtagctctcttaaggaggaacatatctcatctgagacaaacctgaaatgccaaaccaaactgagttagcccctctctgtctgttgtatatattggagtaataacctatttgtcttgataaagggattgcatgcttgaattgcaaaaacctttatttcttttgggttgcccaatgtgcaagactaagagttattttgataaatttctcaccaggctgactgtctctctgtggggtcgggggagttttcagggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaaggtactcactgcttcctgctccccagtactgagcccagaataaaagacgatctcaggctaggagctcaggcaacatcttagtccggtctcatctgttcctggatgtccctcagacccccagctttcatcttttaggatttattccttccctgggataatataatttgtggtccaaaaagaacatcatcaaaatttcaggcagaatgggccaggaaggccattctttcttgatgagtgtccccaaatcatctccaattaacagacaaggagcttgaggttagggaggtgagggtaacactgtctgtaagaggcagagctgggactcaaattccagatttcagattccaaatcccatcgttttttatctctacaatgatgcctcccatctgggtggtggagagaagggaggcgtgtaaaatgtcagccccagaaggacaagagcaagccagtgtgagcggaattgatggctgcaagctgagacttggattggagacgtagtgagactcaggattgtgcagtgctgcagggaagtggttgctggatagaggcatgggctgaaccaagcagctggactgagactgggggacagaactccaaagcccactgagatgtgggaaaacatggagaagcacacggagcattcacaacttattgccgtcagagtcaatacatgggtgaggtggggattgggcaagagggaaagcgtcagccttccctgatattctggaaagtctcccggggctgggggtgggcaggtacagagcttcgagctctgctgatcgctgacatccaggggtgggggtaggaagagacctgggccgggagaagtccacctcaagcctgcagtgtcacactctatccctccacagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataacagagaatcctttttttaacggtgatgcgctgtagaaatgtgactagattttctcattggttctgccctcaagcactgaattcatctgaaactcttggtttcccctggaggccatggttcctgggcaccttgacctgggcaatcccaagtgtggcctgaaccccctttcccttggggattgttcaggtgtccctagacgccttgtggtattgtacctaatacccatgaagggagaggatgatattacttgccagtgtacacccccctgtgatattgttcataatgtccagagtgaagaaagatgatattactcccaatatcacagaaggtgtacacccccccttgatattgttcctaatacccagttggggaggggagaatatctctctcaatatacaaggggtgtttaaactctctgtgatattgttcctaatattcaggggggacaaggatgatattacccaaatattgcaggggttgtacaccccccctttgatattgttcctaatatccaggggtggagaggatattactcccaatattgcaggggtctacatcctccccccgtgacattgttcttaataaccaaaaggtgagaagctgacattactcccaataccacagggggtgtacaccccctatgagatattgttcttaatatccaggaggggagaaaatgatattactctcaatagcgcagggaatttacatcccccgtcgtaatcttgttcttaatattcaggaagggagaggatgatacgactcccagtatcgcagggggtgtgcacccccccgtgatttttttgctaatatccagggtgggagtggataatacgcaggaagtgtacaggtctctgtgatatttttcctaatatccaggggggagaggaagatattacttttaatagtgtacggggggtgtacacccctctgtgatagtgttc

SEQ ID NO: 2 - DNA, 12547 bp, Homo sapiens
gatcagataattgtttagttatgccttcaaaaatgtctgagccaggaacagtggataaatgagcttgtgaatcctcgaaaatttgctctttaagttttgaaatatccaaggttaagttatcatcccaggcttttaaatgtcttgagacattttcccagctatgttgatatttattataagcataaggcattatgcaataatcagaagtattccaatcactctgtaattgcatacggtgttccaaattcatatctcccagccagattacactttggcggagatcattaatttgattagctaatttttgatcaatttgagcctgagaattccaaagtctggaggagtttttctgccatgcttcaattcaacatattgaacggtttgaacagaattgtggatggcaactccagcagttgctgctgttgcagtaaccgcaattagtcctgcaatgatggcaataagagtaaaaataaatctctttgttcttttgcggatacctttaagaacttcattgactatgtgaatagagggggaagactcccatggacgatgtaaagaaactggtatccataccccctccctagcccttaccaagagagtacttgtagtgggattaaaagtagcatcaatgcacgtgaacagcttacagttatcacattctatagtttgtgtattgggaatgataattatatttccaaccaacagcatgtaagggggtttgacatagctcctgatgggtatcacccgttcagatatgagggtgatgttgaatgtgggtgttttggtattagtgtgaaggagttgataggtagtgttccatatccttattcctgtcatagctgcagctaattttcatagttgaggatgttctggggtaacaaagggatgaatcatttttggtctaggaggagtaatgcctgcatccatccatttaaataggtaaggggacacccattgtctcagcctgtatgactgccatccatcctctacataatctaacaaataattaaactctgagcatgaggtatttttgctggagcaatcttgccaataatgcccttttggagcccaatcaataacaatacctgcggctagactttggagcacaacggccttaggagcattacaattattccataaaattgaagtcactataaaaggtccctttgtaggtttctttaaccaaaagagaggctgggcacagttgctcacacttgtaaacccagcactttgggagaccgaggcgggcagatcacgaggtcaggagttcaaaaccagcttggccaacatggtgaaacccctgcctcagcttcctgagtgctgggattataggcatgtgccaccacgcgcagctaattcttgtattttttcagtagagacgaggtttcatcatgttgcccaggctggtctcgagtacctgacctcaacctgaggtgatccaaccacctcagcctcccaaagtgctgggattacaagcatgagtcaccgcacccgggccccagtcactttagaatagcatgttgctttgtattcggagggtctctctgcaaatagcccatcaacactgagcgtgcctggaaagacctggttttcaaataactggcttcgtctgtgtaaaacgagtcttgttgtatgcattaaaaattatcttggctgggcgtggtggctcacgcctttaatcccagcattttgggaggctcgttctgttgccagggaggctgcaaaaggaggatgcggctatttctgcttccatcatggaaggctgggattccacaggcacagaaaggaaggagggagagatgggaatgtgactgtcctccagacacagcctctggcaggatcggatgtgggagagtgagggtcccaccccagctggggtctacccaggtccatgtcttggacatgttgagagtttttctggaaggcagggatacagtgtggtccaaaaacacacaaatgcccctactggcccaggggttgtcacaatagactggaagggtgacacatcccaggcgcttgccacccatcacacgcacctcctacccactggcatccttccaccccaggcacacacaaagcctcagtccagagatcaactctggactcagctctgaatttgcatatcctgtgtgtagattcattcttcataacctctgcccagcctagcttgtgtatcatttttttttctctattaggggaggagcccgtcctggcactcccattggcctgtagattcacctcccctgggcagggccccaggacccaggataatatctgtgcctcctgcccagaaccctccaagcagacacaatggtaagaatggtgcctgtcctgctgtctctgctgctgcttctgggtcctgctgtcccccaggagaaccaagatggtgagtggggaaagcaagggatgggtgctggagaggactggaaggaggtgaggaacaggacatgtggctgggagacaggctggatgcagctgggataccctggcatacggcaggaatgggtgcccaaggctgtcaactccctcagctcacacacttccaggagcattcagggagcctctgcgctggcccgaaataagaccttcaggaatctgaatctaaaacccctagtttacagtgaaaacaaagactccaaagaccaagcgacctgcttggggtagacagtcaggacggagtaggaaccatatgcctggagctgcttctgctcctgttccttccctccttccgatggctgggtacacctgcctgacgctgatgaaaagagagagcagccccaaggggaaagtgggaaggcaggttggctggagggatggtgctagaaggaaacccgtgcccaaatcccacactcagacaccactgcagtgggtctggaaggcgagtggctggaagagaagagagtgggagctccgggagatcaagagtcactcctaggataagggaaggaggctgtttgtggcatgagaatgtgcaggataaagacatggaagcgaatggcttctcagttgtgtgagtttaaaattcatgacatttacaaattgtcagaaaaggtgttatatgtttgttatataacaatcactttggaatgttaatctgattctgtgccaaaatctgaattactcagggttctccagagaaacagaactaataggtggtacacatatacatatatatgtacgtacacatacatacatacactgtatacacatggatacacacacacataggaagagatttacatatatgtatacaaaagagagagagagtagagatttattttaagaaattgactcacactattgggaggagtaacaagtcctaaatcttcagagccggccagcaggctggagacccagggaagagttgatgtcttagtcttgattccaagggcagactgtaggcagaattctttcctctttaggggacatctgaggctttttctcttaaggccttcaactgattggatgaagcccaccactatggagagtaatccactttactcaaggtctactgatttttttgtaaattaaaaaaaaaactgtgggtgcatagtatgtgtatatatttatggggtacatgagaggttttgattcaggcatgcaatgtgaaataatcacatcatcaaaaatgaggtatccatcccttcaagcttttatcgtttgtgttacagacaatccaattatacttttttggttattttagtttttaaaagtatttgattatttatttatttatttatttttgagacagagtctcactctgtcacccaggcaggagtgcagtggcatgatctcggctcactgcaacctccgcctcccaggttcaagcaattttcctgcctcagtctcctgagtagctaggactacaggcacctgccaccacacctggctaatttttttgtatttttagtagagacgggtttcatcatgttggccaggctagtcttgatatcctgacctcgtgatctgcccgccttggtctcccaaagtgccgggattacaggtgtcagcaactgcgcctggcctctcttttggttatttaaaagtgtacaattaaattatgattattattattatttttgagatggattcttgttctgtcacccaggctggagtgcagtggcgtgatcttggcttactgcaaacctccgcctgttgggttcaagcaattatcttgcctcgggtgtacactgccacacacggctaacttatgtatttttaatagagatagggtttcaccatgttggctagactggtcttgacctcttgacctcaagtgatccactcacttcagcctcccagagtgctggaattacaggcacgagccaccacacctggccccagttaaattattattgactatagtcaccctgttgtgctatcaaatagtaggtcttattcattcttctttttttttttttttttgtgacagagttgcccaggctggaatgcagtggtgcaatcttggctcactgcaacctctgcctcccgggcttaagcgattctcctgcctcagccttctgagtcgctgggactacaggtgtgtgccaccacgcccggctaatttatgtatttttagtagagatggggtttcaccatgttggccaggctggtttcgaactcctgacctcaagtgacccacctgcctcagcttcccaaagtgttggaattacaggcatgagccaccacacctggccccagttaaattattattcactggagtcactttgttgtgctatcaaatagttttctaactattttttttgtacccattaaccaccctcccaatttccccccaaccctgccactacccttcccagcctttggtaaccatccttctactctctatgtccatgaattcaattgtagggtctactgatttaaaggctaatcacatttagacactcaggagcaagaataattttagtaattgaactaggattctgccatatgacctccaacatcattagcacctgtgtaaattgtatcataaaataattatggaactattatggaaatgtccctctctcccagatcccaccttgtaccaaaatgcaaggtacaaccccgggaattctgagctccatcctagtcttaccctgtgctaattcagtctgggtcatttcttgaattttctggtaaattctcctttctaccctttctaactatatgtatttgtcaggttaagctagaagtgttaattttttttttttttgagatggagccttgctttgtcacctaggctgaagtgcagtggcatgatctcagctcactgcaagctccgcctcccgggttcatgccattctcctgcctcagcctcctgagtagctgggactacaggcacccgccaccatgcttggctaattttgtgaattcttagtagagacggggtttcaccatgttagccaggatggtctcgatctcctgacctcgtgatccacccgcctcggccccctaaagtgctgggattacaggcgtgagccactgagcccggacgaaatgttaatttgttttttttgagacggagtctcactctgtcatccaagctggagtgcagtggcatgatcttggcttgttgcaacctctgcctctctggttcaagtgattttcctgcctcagcctccagcatgactgggattacaggcccgcaccaccatgcccagctaatttttgtattttttaatagagatggggtttcaccatgttggccaggctggtcttcaactcctgatctcaagtaatctgcctgccttggcctcccaaagtcctgggattacaggcatgagccacggagcccagcctagaaatgttaatttctaacgcatgtcagattccatgcacactgggcaaggttccattcctccatggggtgactcagggatccaggccaattgcatattgagactctttcatattatcctgtggccttcaaagtcgtcacctctagggatgagaaacaaaagggacaagccagctggtagggtcttggacaagaagaaagacatcacttctgctcacattctcttttgacaaaactcagtcacatggtcccaatatatcttcgaggtggctgagtaatgttatcttcctatgtgtcaagcagaggaaataatgtagtgaagacacaggatggtctctgaaatatcatctcaggcatgaaagtagagcatattcacttgagtgagcctccagtggtgtgaagttgatggcaggagaaagagctggggaagaaaaggccagtggcaggtctcccctcctagccctatgcagccccacagtgggacccttgcatggacctcaaccatcagaatcttttcttttgcaggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacatcgtggagtattacaacgacagtaacggtcagtgaataacagaccacaggggtggaaggtctaacccaagaggcagcccccccagtgtgagtggcaagggatcagcaggatggaaatagtcccaatcccaggggaagaacaggagacacagcagaaacacagacatgtccacatcccacccaccccacagcacaggtgctccccgcttccccatcaattgccccatcctcatcccaggcctcaggtcacacaggaagtgatggcagagtcacttcctatccaggcacctatgacctctcacctccacaccccacccatcggaggctgatacccccgtgagaaggcatcagactcacccctgtccagggaggttgcctggagagtgagccactctcaaagtcactcagacctgggctcacctggtggttctgccagtcctagctgttgacagtgaaacgttcccaaaatatctggttgaaatctgcaaacattggagcactgagacctacctccaaacaagtctgtaatatttaactatgtctgttctatgaaggatgtcacagtctgtcctgatctcccttgcagctccatcacctagcacagggtacagccaatattggctcaattgaaatttgtggaatccacagagaaaagcacccggcacacaccgtagcccatgctgggggctcaggaagtgctggattcaaaactgtgggctgttagagttccttggagccctaaagttcctccttaccatacgatgcagacccaggaagggccacctgcgctatggtcagaggagctggtggcagagcccgtgcagagatggtccctgtgcccccggcccagtgctctttctcctaaaccacactgccagccccaaggcagccaacctcaggtctggtgaactgctggtgttaaattatcatagagtgggtgtcaaaagatgggctactaagtacaaaaatgcccaaggtgctacatgggatctgaagattttcaaaaggaggcaagaaagagataggcagatgtttcaaggatgtggggtgggggaggtcttggtaaggaaaatggcccaggctgtgtgtcagcaataggagaggagggggcacaggtgatcagaaaagacactgggggaagcattgatggacaggaatagaaatggcaaagtggataattaagaggaaggaggatgaggagatgaacacagggtattagaaaataatagaaggcagggcttggtggctcactcttgtaatcccagcactttgggaggctgaggcaggcagatcacctaaggtcaggagttcgagaccagcccggccaacatggtgaaaccctgtctctactaataatacaaaaatagcctggcatggtggcacacgtctgtggtcccagctactcaggaggctgaggcaggagaattgcttgaacccaggaggcagaggttacagtgagccaaaatcctaccattgcactacagcctgagtgacaagagtgaaacgttgtctaaaaacaaaaaacaaaaaacaaaaaaaggaaataatagtagctgacatttactgagcacttactttgtgccaggcccatctatgagcatatataatgctcagaatagccccctaaaacagtgctcttggcattgccatttcagaggtgaggaaatagaggcacagggagttgagtggctccagttcaggcaacacaccaggtgggggtggggggctggggagagacctgggacgtgagcccagacagcttgagagctttcagagtctatgccaacagcaccaaccagtgctgggtaaacacctgcttttatcatcagaacaaagaggctgtgtcccctgccctatgaggtccatttctgagagttgtggctaatgggcaagaaggttggggctttagagatttgggataaagatatcaaacaccagaaaagtagaaagaagtgatcagattagggttacttaggtgatgatatgaactcttcctagaactgagagaaaaagagagccttcctttactcatatgaaatcacaaataatttctatccaatttggaagtacactttggtgtagttgtgacagcttcctcaggactcagcataaattcaaacaaataattgtccttagaagagatgctatagaagagatagaaatatattcatattctgtagcttttttttttttgagatggatttttgctcttgtcacccaagctggagtgcagtgatgcaatctcagctcactgcaaactttgcctcctgggttcaagggattctcctgcctcagcctcccgataactgggactacaggctacaggcatgtgtcactactcctggttaatttttttttttttttttaagactgagtcttgctctgtctttcaggctgatgtacaatggctccatctcggctcactacaacttctgtcccccaggttcaagcgattctcctgcctcagcctcatgagtagctgggattacaggcatgtgccagcacacccagcaaatttttgtatttttagtagagatgaggtcttaccatgttggccaggctggtctcaaactcctgacctcaggtgatcctttggcctcagcctccctaactgctgggattacaggcatgagccactgcgtccagcctaattttatatttttggtagagatggggtttcaccatattggccaggctggtctcgaactcatgacctaaggtgatccatcctcctcagcctctcaaagtgctgggattacaagtgtgagccactgggcctggtgctttttttttttttttttttttttttttttttttttgagatagggtctcactctgtcacccaggctgaaatgcagtagtgtgattttggctcattgcagccttgacttcccaggctgaagtgatcctcccacctcagcctcctgagtagctggggctacaggcatgcaccaccatgctgcgctaatttttatattttttgtagtggtgggattttgccatatcaccctggctggtctggaacccctgggctcaagcgatccactcgcttcagcttctcaaagtgctgggattacaggcatgagccacagcgcccaggctgtagctctcttaaggaggaacatatctcatctgagacaaacctgaaatgccaaaccaaactgagttagcccctctctgtctgttgtatatattggagtaataacctatttgtcttgataaagggattgcatgcttgaattgcaaaaacctttatttcttttgggttgcccaatgtgcaagactaagagttattttgataaatttctcaccaggctgactgtctctctgtggggtcgggggagttttcagggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaaggtactcactgcttcctgctccccagtactgagcccagaataaaagacgatctcaggctaggagctcaggcaacatcttagtccggtctcatctgttcctggatgtccctcagacccccagctttcatcttttaggatttattccttccctgggataatataatttgtggtccaaaaagaacatcatcaaaatttcaggcagaatgggccaggaaggccattctttcttgatgagtgtccccaaatcatctccaattaacagacaaggagcttgaggttagggaggtgagggtaacactgtctgtaagaggcagagctgggactcaaattccagatttcagattccaaatcccatcgttttttatctctacaatgatgcctcccatctgggtggtggagagaagggaggcgtgtaaaatgtcagccccagaaggacaagagcaagccagtgtgagcggaattgatggctgcaagctgagacttggattggagacgtagtgagactcaggattgtgcagtgctgcagggaagtggttgctggatagaggcatgggctgaaccaagcagctggactgagactgggggacagaactccaaagcccactgagatgtgggaaaacatggagaagcacacggagcattcacaacttattgccgtcagagtcaatacatgggtgaggtggggattgggcaagagggaaagcgtcagccttccctgatattctggaaagtctcccggggctgggggtgggcaggtacagagcttcgagctctgctgatcgctgacatccaggggtgggggtaggaagagacctgggccgggagaagtccacctcaagcctgcagtgtcacactctatccctccacagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataacagagaatcctttttttaacggtgatgcgctgtagaaatgtgactagattttctcattggttctgccctcaagcactgaattcatctgaaactcttggtttcccctggaggccatggttcctgggcaccttgacctgggcaatcccaagtgtggcctgaaccccctttcccttggggattgttcaggtgtccctagacgccttgtggtattgtacctaatacccatgaagggagaggatgatattacttgccagtgtacacccccctgtgatattgttcataatgtccagagtgaagaaagatgatattactcccaatatcacagaaggtgtacacccccccttgatattgttcctaatacccagttggggaggggagaatatctctctcaatatacaaggggtgtttaaactctctgtgatattgttcctaatattcaggggggacaaggatgatattacccaaatattgcaggggttgtacaccccccctttgatattgttcctaatatccaggggtggagaggatattactcccaatattgcaggggtctacatcctccccccgtgacattgttcttaataaccaaaaggtgagaagctgacattactcccaataccacagggggtgtacaccccctatgagatattgttcttaatatccaggaggggagaaaatgatattactctcaatagcgcagggaatttacatcccccgtcgtaatcttgttcttaatattcaggaagggagaggatgatacgactcccagtatcgcagggggtgtgcacccccccgtgatttttttgctaatatccagggtgggagtggataatacgcaggaagtgtacaggtctctgtgatatttttcctaatatccaggggggagaggaagatattacttttaatagtgtacggggggtgtacacccctctgt

SEQ ID NO: 3 - DNA, 31 bp, Homo sapiens
aataacaatacctgcggctagactttggagc

SEQ ID NO: 4 - DNA, 31 bp, Homo sapiens
aaccaaaagagaggctgggcacagttgctca

SEQ ID NO: 5 - DNA, 31 bp, Homo sapiens
acagttgctcacacttgtaaacccagcactt

SEQ ID NO: 6 - DNA, 31 bp, Homo sapiens
gcatgtgccaccacgcgcagctaattcttgt

SEQ ID NO: 7 - DNA, 31 bp, Homo sapiens
taggaaccatatgcctggagctgcttctgct

SEQ ID NO: 8 - DNA, 31 bp, Homo sapiens
cctgcctgacgctgatggaaagagagagcag

SEQ ID NO: 9 - DNA, 31 bp, Homo sapiens
tgcctgacgctgaggaaaagagagagcaccc

SEQ ID NO: 10 - DNA, 31 bp, Homo sapiens
tcagccttctgagtcgctgggactacaggtg

SEQ ID NO: 11 - DNA, 31 bp, Homo sapiens
attatggaactattatggaaatgtccctctc

SEQ ID NO: 12 - DNA, 31 bp, Homo sapiens
tgcttggctaattttgtgaattcttagtaga

SEQ ID NO: 13 - DNA, 31 bp, Homo sapiens
gaccctgaaagacatcgtggagtattacaac

SEQ ID NO: 14 - DNA, 31 bp, Homo sapiens
aacacagacatgtccacatcccacccacccc

SEQ ID NO: 15 - DNA, 31 bp, Homo sapiens
ggaggctgatacccccgtgagaaggcatcag

SEQ ID NO: 16 - DNA, 31 bp, Homo sapiens
gaaatttgtggaatccacagagaaaagcacc

SEQ ID NO: 17 - DNA, 31 bp, Homo sapiens
cagagaaaagcacccggcacacaccgtagcc

SEQ ID NO: 18 - DNA, 31 bp, Homo sapiens
ccaaggcagccaacctcaggtctggtgaact

SEQ ID NO: 19 - DNA, 31 bp, Homo sapiens
gcagccaacctcaggtctggtgaactgctgg

SEQ ID NO: 20 - DNA, 31 bp, Homo sapiens
ttgcactacagcctgagtgacaagagtgaaa

SEQ ID NO: 21 - DNA, 31 bp, Homo sapiens
ttgtctaaaaacaaaaaacaaaaaacaaaaa

SEQ ID NO: 22 - DNA, 30 bp, Homo sapiens
atcaaacaccagaaaagtagaaagaagtga

SEQ ID NO: 23 - DNA, 31 bp, Homo sapiens
gtagtggtgggattttgccatatcaccctgg

SEQ ID NO: 24 - DNA, 31 bp, Homo sapiens
tgcttcctgctccccagtactgagcccagaa

SEQ ID NO: 25 - DNA, 31 bp, Homo sapiens
catctccaattaacagacaaggagcttgagg

SEQ ID NO: 26 - DNA, 31 bp, Homo sapiens
gtccacctcaagcctgcagtgtcacactcta

SEQ ID NO: 27 - DNA, 31 bp, Homo sapiens
gggagaatatctctctcaatatacaaggggt

SEQ ID NO: 28 - DNA, 31 bp, Homo sapiens
tcccagtatcgcagggggtgtgcaccccccc

SEQ ID NO: 29 - DNA, 21 bp, Artificial Sequence
  Description of Artificial Sequence Synthetic primer
gtccaaaaacacacaaatgcc

SEQ ID NO: 30 - DNA, 20 bp, Artificial Sequence
  Description of Artificial Sequence Synthetic primer
ttcctcacctccttccagtc

SEQ ID NO: 31 - DNA, 20 bp, Artificial Sequence
  Description of Artificial Sequence Synthetic primer
tccaaccaacagcatgtaag

SEQ ID NO: 32 - DNA, 20 bp, Artificial Sequence
  Description of Artificial Sequence Synthetic primer
ccctccgaatacaaagcaac

SEQ ID NO: 33 - DNA, 20 bp, Artificial Sequence
  Description of Artificial Sequence Synthetic primer
agaaccctccaagcagacac

SEQ ID NO: 34 - DNA, 24 bp, Artificial Sequence
  Description of Artificial Sequence Synthetic primer
ggcacagaatcagattaacattcc

SEQ ID NO: 35 - DNA, 21 bp, Artificial Sequence
  Description of Artificial Sequence Synthetic primer
ttctaacgcatgtcagattcc

SEQ ID NO: 36 - DNA, 21 bp, Artificial Sequence
  Description of Artificial Sequence Synthetic primer
ctatttccatcctgctgatcc

SEQ ID NO: 37 - DNA, 20 bp, Artificial Sequence
  Description of Artificial Sequence Synthetic primer
tgagacaaacctgaaatgcc

SEQ ID NO: 38 - DNA, 19 bp, Artificial Sequence
  Description of Artificial Sequence Synthetic primer
aagcagtgagtaccttgcc

SEQ ID NO: 39 - DNA, 20 bp, Artificial Sequence
  Description of Artificial Sequence Synthetic primer
aagagcaagccagtgtgagc

SEQ ID NO: 40 - DNA, 22 bp, Artificial Sequence
  Description of Artificial Sequence Synthetic primer
aaatccacctcctgtctgtccc

SEQ ID NO: 41 - DNA, 19 bp, Artificial Sequence
  Description of Artificial Sequence Synthetic primer
agcagcccagataaccaag

SEQ ID NO: 42 - DNA, 21 bp, Artificial Sequence
  Description of Artificial Sequence Synthetic primer
gcaataagttgtgaatgctcc

SEQ ID NO: 43 - DNA, 20 bp, Artificial Sequence
  Description of Artificial Sequence Synthetic primer
gctcactacaacttctgtcc

SEQ ID NO: 44 - DNA, 22 bp, Artificial Sequence
  Description of Artificial Sequence Synthetic primer
ggcaacccaaaagaaataaagg

SEQ ID NO: 45 - DNA, 19 bp, Artificial Sequence
  Description of Artificial Sequence Synthetic primer
agttcaggcaacacaccag

SEQ ID NO: 46 - DNA, 20 bp, Artificial Sequence
  Description of Artificial Sequence Synthetic primer
ggccaacatggtaagacctc

SEQ ID NO: 47 - DNA, 20 bp, Artificial Sequence
  Description of Artificial Sequence Synthetic primer
ggcaagaaagagataggcag

SEQ ID NO: 48 - DNA, 21 bp, Artificial Sequence
  Description of Artificial Sequence Synthetic primer
ccacaactctcagaaatggac

SEQ ID NO: 49 - DNA, 20 bp, Artificial Sequence
  Description of Artificial Sequence Synthetic primer
agccactctcaaagtcactc

SEQ ID NO: 50 - DNA, 22 bp, Artificial Sequence
  Description of Artificial Sequence Synthetic primer
agccctgccttctattattttc

SEQ ID NO: 51 - DNA, 20 bp, Artificial Sequence
  Description of Artificial Sequence Synthetic primer
acaggtggaaggaatggagg

SEQ ID NO: 52 - DNA, 20 bp, Artificial Sequence
  Description of Artificial Sequence Synthetic primer
taggtgatggagctgcaagg

SEQ ID NO: 53 - DNA, 22 bp, Artificial Sequence
  Description of Artificial Sequence Synthetic primer
cttaccctgtgctaattcagtc

SEQ ID NO: 54 - DNA, 21 bp, Artificial Sequence
  Description of Artificial Sequence Synthetic primer
gtcccttttgtttctcatccc

SEQ ID NO: 55 - DNA, 20 bp, Artificial Sequence
  Description of Artificial Sequence Synthetic primer
tacccattaaccaccctccc

SEQ ID NO: 56 - DNA, 21 bp, Artificial Sequence
  Description of Artificial Sequence Synthetic primer
gcttggatgacagagtgagac

SEQ ID NO: 57 - DNA, 20 bp, Artificial Sequence
  Description of Artificial Sequence Synthetic primer
ggattcttgttctgtcaccc

SEQ ID NO: 58 - DNA, 22 bp, Artificial Sequence
  Description of Artificial Sequence Synthetic primer
cttgctcctgagtgtctaaatg

SEQ ID NO: 59 - DNA, 20 bp, Artificial Sequence
  Description of Artificial Sequence Synthetic primer
ggatgaagcccaccactatg

SEQ ID NO: 60 - DNA, 20 bp, Artificial Sequence
  Description of Artificial Sequence Synthetic primer
ggtcaagaggtcaagaccag

SEQ ID NO: 61 - DNA, 20 bp, Artificial Sequence
  Description of Artificial Sequence Synthetic primer
cccaaatcccacactcagac

SEQ ID NO: 62 - DNA, 20 bp, Artificial Sequence
  Description of Artificial Sequence Synthetic primer
agcttgaagggatggatacc

SEQ ID NO: 63 - DNA, 21 bp, Artificial Sequence
  Description of Artificial Sequence Synthetic primer
cacaatggaaatggcacttac

SEQ ID NO: 64 - DNA, 20 bp, Artificial Sequence
  Description of Artificial Sequence Synthetic primer
gatattctcccctccccaac

SEQ ID NO: 65 - DNA, 18 bp, Artificial Sequence
  Description of Artificial Sequence Synthetic primer
tgaaccccctttcccttg

SEQ ID NO: 66 - DNA, 18 bp, Artificial Sequence
  Description of Artificial Sequence Synthetic primer
atcttcctctcccccctg

SEQ ID NO: 67 - DNA, 4600 bp, Homo sapiens
gtcccagctactcaggaggctgaggcaggagaattgcttgaacccaggaggcagaggttacagtgagccaaaatcctaccattgcactacagcctgagtgacaagagtgaaacgttgtctaaaaacaaaaaacaaaaaaaggaaataatagtagctgacatttactgagcacttactttgtgccaggcccatctatgagcatatataatgctcagaatagccccctaaaacagtgctcttggcattgccatttcagaggtgaggaaatagaggcacagggagttgagtggctccagttcaggcaacacaccaggtgggggtggggggctggggagagacctgggacgtgagcccagacagcttgagagctttcagagtctatgccaacagcaccaaccagtgctgggtaaacacctgcttttatcatcagaacaaagaggctgtgtcccctgccctatgaggtccatttctgagagttgtggctaatgggcaagaaggttggggctttagagatttgggataaagatatcaaacaccagaaaagtagaaagaagtgatcagattagggttacttaggtgatgatatgaactcttcctagaactgagagaaaaagagagccttcctttactcatatgaaatcacaaataatttctatccaatttggaagtacactttggtgtagttgtgacagcttcctcaggactcagcataaattcaaacaaataattgtccttagaagagatgctatagaagagatagaaatatattcatattctgtagcttttttttttttgagatggatttttgctcttgtcacccaagctggagtgcagtgatgcaatctcagctcactgcaaactttgcctcctgggttcaagggattctcctgcctcagcctcccgataactgggactacaggctacaggcatgtgtcactactcctggttaatttttttttttttttttaagactgagtcttgctctgtctttcaggctgatgtacaatggctccatctcggctcactacaacttctgtcccccaggttcaagcgattctcctgcctcagcctcatgagtagctgggattacaggcatgtgccagcacacccagcaaatttttgtatttttagtagagatgaggtcttaccatgttggccaggctggtctcaaactcctgacctcaggtgatcctttggcctcagcctccctaactgctgggattacaggcatgagccactgcgtccagcctaattttatatttttggtagagatggggtttcaccatattggccaggctggtctcgaactcatgacctaaggtgatccatcctcctcagcctctcaaagtgctgggattacaagtgtgagccactgggcctggtgctttttttttttttttttttttttttttttttttttgagatagggtctcactctgtcacccaggctgaaatgcagtagtgtgattttggctcattgcagccttgacttcccaggctgaagtgatcctcccacctcagcctcctgagtagctggggctacaggcatgcaccaccatgctgcgctaatttttatattttttgtagtggtgggattttgccatatcaccctggctggtctggaacccctgggctcaagcgatccactcgcttcagcttctcaaagtgctgggattacaggcatgagccacagcgcccaggctgtagctctcttaaggaggaacatatctcatctgagacaaacctgaaatgccaaaccaaactgagttagcccctctctgtctgttgtatatattggagtaataacctatttgtcttgataaagggattgcatgcttgaattgcaaaaacctttatttcttttgggttgcccaatgtgcaagactaagagttattttgataaatttctcaccaggctgactgtctctctgtggggtcgggggagttttcagggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaaggtactcactgcttcctgctccccagtactgagcccagaataaaagacgatctcaggctaggagctcaggcaacatcttagtccggtctcatctgttcctggatgtccctcagacccccagctttcatcttttaggatttattccttccctgggataatataatttgtggtccaaaaagaacatcatcaaaatttcaggcagaatgggccaggaaggccattctttcttgatgagtgtccccaaatcatctccaattaacagacaaggagcttgaggttagggaggtgagggtaacactgtctgtaagaggcagagctgggactcaaattccagatttcagattccaaatcccatcgttttttatctctacaatgatgcctcccatctgggtggtggagagaagggaggcgtgtaaaatgtcagccccagaaggacaagagcaagccagtgtgagcggaattgatggctgcaagctgagacttggattggagacgtagtgagactcaggattgtgcagtgctgcagggaagtggttgctggatagaggcatgggctgaaccaagcagctggactgagactgggggacagaactccaaagcccactgagatgtgggaaaacatggagaagcacacggagcattcacaacttattgccgtcagagtcaatacatgggtgaggtggggattgggcaagagggaaagcgtcagccttccctgatattctggaaagtctcccggggctgggggtgggcaggtacagagcttcgagctctgctgatcgctgacatccaggggtgggggtaggaagagacctgggccgggagaagtccacctcaagcctgcagtgtcacactctatccctccacagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataacagagaatcctttttttaacggtgatgcgctgtagaaatgtgactagattttctcattggttctgccctcaagcactgaattcatctgaaactcttggtttcccctggaggccatggttcctgggcaccttgacctgggcaatcccaagtgtggcctgaaccccctttcccttggggattgttcaggtgtccctagacgccttgtggtattgtacctaatacccatgaagggagaggatgatattacttgccagtgtacacccccctgtgatattgttcataatgtccagagtgaagaaagatgatattactcccaatatcacagaaggtgtacacccccccttgatattgttcctaatacccagttggggaggggagaatatctctctcaatatacaaggggtgtttaaactctctgtgatattgttcctaatattcaggggggacaaggatgatattacccaaatattgcaggggttgtacaccccccctttgatattgttcctaatatccaggggtggagaggatattactcccaatattgcaggggtctacatcctccccccgtgacattgttcttaataaccaaaaggtgagaagctgacattactcccaataccacagggggtgtacaccccctatgagatattgttcttaatatccaggaggggagaaaatgatattactctcaatagcgcagggaatttacatcccccgtcgtaatcttgttcttaatattcaggaagggagaggatgatacgactcccagtatcgcagggggtgtgcacccccccgtgatttttttgctaatatccagggtgggagtggataatacgcaggaagtgtacaggtctctgtgatatttttcctaatatccaggggggagaggaagatattacttttaatagtgtacggggggtgtacacccctctgtgatagtgttc

(19) United States
(12) Patent Application Publication
(10) Pub. No.: US 2010/0086912 A1
(43) Pub. Date: Apr. 8, 2010

(54) AZGP Gene Single Nucleotide Polymorphisms (SNPs)

(76) Inventors: Roger G. Clerc, Binningen (CH); Guillemette Duchateau-Nguyen, Riedisheim (FR); Laurent Essioux, Attenschwiller (FR); Delphine Lagarde, Hegenheim (FR); Claes-Goran Ostenson, Solna (SE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/084,743

(22) PCT Filed: Nov. 9, 2006
(86) PCT No.: PCT/EP2006/010726

§ 371 Date: May 9, 2008

(30) Foreign Application Priority Data
Nov. 15, 2005 (EP) 05110738.1

(51) Int. Cl. C12Q 1/68; C07H 21/04

(52) U.S. Cl. 435/6; 536/23.1

(57) ABSTRACT

The present invention provides single nucleotide polymorphisms and haplotypes in the AZGP1 gene that can be used for determining the predisposition of an individual to obesity.

The present invention relates to SNPs and haplotypes in the AZGP1 gene associated with obesity, and methods for determining predisposition of an individual to obesity by the presence or absence of said SNPs and/or haplotypes in the AZGP1 gene.

Multifactorial diseases such as obesity are caused by mutations in more than one gene with a large contribution from environmental factors. There has been spectacular success in identifying the genes responsible for Mendelian disorders, whereas finding the susceptibility genes involved in multifactorial diseases has so far been difficult. The evidence suggests that humans inherit a genetic predisposition to gain weight on a high fat diet. It would be useful to identify markers of predisposition of individuals to obesity.

AZGP1 is a Zn-Alpha2-glycoprotein the gene of which is down-regulated in obesity (EP 1548445), and up-regulated in cachexia (Russell and Tisdale, 2005, Brit. J. Cancer 92, 876-881; Russell et al., 2004, Biochem. Biophys. Acta 1636, 59-68; Sanders and Tisdale, 2004, Cancer Lett. 212, 71-81; Bing et al., 2004, Proc. Natl. Acad. Sci USA 101, 2500-2505).

So far, no AZGP1 haplotypes have been associated with obesity.

### DESCRIPTION OF THE INVENTION

The problem to be solved by the present invention was to provide markers for the predisposition of individuals to obesity. The problem was solved by the present invention by the identification of SNPs and haplotypes in the AZGP1 gene which are associated with obesity. DNA samples obtained from lean and obese subjects were used to identify haplotypes and SNPs in the AZGP1 gene. These SNPs and haplotypes were associated with obesity. As it is known from the literature that obesity is associated with insulin resistance, these SNPs may also be linked to insulin resistance. Obese subjects who participated in this study were non-diabetic when the samples were taken. DNA fragments of the AZGP1 gene were amplified by PCR and sequenced. Following sequencing, polymorphism analysis was performed using the Polyphred software (University of Washington). Table 1 lists all markers identified in AZGP1.

Table 2 is showing the allele frequency of all polymorphic sites found in AZGP1 DNA samples. For haplotype frequency calculations, only SNPs with a minor allele frequency higher than 5% were used. The less frequent markers are not likely to be selected in further association studies and will not contribute substantially to the common haplotypes. Out of the 28 markers presented in Table 2, 15 (in bold) were further included in the haplotype analysis.

Table 3 is providing the haplotype frequencies on the 15 frequent markers of AZGP1. As can be seen in the table some marker couples were completely redundant (equivalence of occurrence of alleles in the different haplotypes):
zag18 and zag19
zag17, zag16 and the intronic deletion (zag del).
The cluster zag16, zag17 and the intronic deletion (zag del) is nearly redundant with zag15 and zag35. By looking at Table 6, from zag17 to zag35, only 3 haplotypes are present: AdelATT, GwtGCC and GwtGTC. The last haplotype is only present in H12 which is a rare haplotype (f=0.005).

TABLE 1

Characteristics of all markers
identified in AZGP1, in DNA samples

| | | | | | |
|---|---|---|---|---|---|
| | | | | | Seq |
| | | | | Location | ID |
| Marker | Pos. | Alleles | Flanking sequences | in AZGP1 | No. |

| | | | | | |
|---|---|---|---|---|---|
| zag06 | 1090^{1,2} | G/A | AATAACAATACCTGCGGCTAGACTTTGGAGC | unknown | 3 |
| zag05 | 1196^{1,2} | T/C | AACCAAAAGAGAGGCTGGGCACAGTTGCTCA | unknown | 4 |
| zag04 | 1216^{1,2} | T/A | ACAGTTGCTCACACTTGTAAACCCAGCACTT | unknown | 5 |
| zag03 | 1348^{1,2} | C/T | GCATGTGCCACCACGCGCAGCTAATTCTTGT | unknown | 6 |
| zag07 | 2695^{1,2} | T/C | TAGGAACCATATGCCTGGAGCTGCTTCTGCT | Intron 1 | 7 |
| zag08 | 2760^{1,2} | T/G | CCTGCCTGACGCTGATGGAAAGAGAGAGCAG | Intron 1 | 8 |
| zag09 | 2762^{1,2} | A/G | TGCCTGACGCTGAGGAAAAGAGAGAGCACCC | Intron 1 | 9 |
| zag13 | 4528^{1,2} | C/A | TCAGCCTTCTGAGTCGCTGGGACTACAGGTG | Intron 1 | 10 |
| zag12 | 5013^{1,2} | T/C | ATTATGGAACTATTATGGAAATGTCCCTCTC | Intron 1 | 11 |
| zag10 | 5369^{1,2} | C/T | TGCTTGGCTAATTTTGTGAATTCTTAGTAGA | Intron 1 | 12 |
| zag11 | 6561^{1,2} | C/T | GACCCTGAAAGACATCGTGGAGTATTACAAC | Ex02, silent | 13 |
| zag23 | 6730^{1,2} | A/G | AACACAGACATGTCCACATCCCACCCACCCC | Intron 2 | 14 |
| zag22 | 6894^{1,2} | C/T | GGAGGCTGATACCCCCGTGAGAAGCCATCAG | Intron 2 | 15 |
| zag18 | 7202^{1,2} | C/A | GAAATTTGTGGAATCCACAGAGAAAAGCACC | Intron 2 | 16 |
| zag19 | 7219^{1,2} | G/A | CAGAGAAAAGCACCCGGCACACACCGTAGCC | Intron 2 | 17 |
| zag20 | 7454^{1,2} | T/C | CCAAGGCAGCCAACCTCAGGTCTGGTGAACT | Intron 2 | 18 |
| zag21 | 7459^{1,2} | T/C | GCAGCCAACCTCAGGTCTGGTGAACTGCTGG | Intron 2 | 19 |
| zag17 | 8047^{1,2} | A/C | TTGCACTACAGCCTGAGTGACAAGAGTGAAA | Intron 2 | 20 |
| zag del | 8077 | AAAAAAC/. | TTGTCTAAAAACAAAAAACAAAAAACAAAAA | Intron 2 | 21 |
| | 8083² | | | | |
| zag16 | 8493¹ | A/G | ATCAAACACCAGAAAAGTAGAAAGAAGTGA | Intron 2 | 22 |
| | (8500²) | | | | |
| zag15 | 9549¹ | T/C | GTAGTGGTGGGATTTTGCCATATCACCCTGG | Intron 2 | 23 |
| | (9556²) | | | | |
| zag24 | 10202¹ | A/C | TGCTTCCTGCTCCCCAGTACTGAGCCCAGAA | Intron 3 | 24 |
| | (10209²) | | | | |
| zag25 | 10439¹ | G/A | CATCTCCAATTAACAGACAAGGAGCTTGAGG | Intron 3 | 25 |
| | (10446²) | | | | |
| zag26 | 11020¹ | G/T | GTCCACCTCAAGCCTGCAGTGTCACACTCTA | Intron 3 | 26 |
| | (11027²) | | | | |
| zag35 | 11995¹ | T/C | GGGAGAATATCTCTCTCAATATACAAGGGGT | unknown | 27 |
| | (12002²) | | | | |
| zag34 | 12385¹ | G/T | TCCCAGTATCGCAGGGGGTGTGCACCCCCCC | unknown | 28 |
| | (12392²) | | | | |

¹Position of the marker in the EMBL accession number ac004977.
²Position of the marker in Seq ID No. 2

TABLE 2
Allelic frequency of discovered SNPs in AZGP1.

| | | Distance | | | | | |
|---|---|---|---|---|---|---|---|
| Marker | region | (bp)¹ | All1 | All2 | N² | f_All1 | f_All2 |
| zag06 | 5′reg | — | G | A | 93 | 0.995 | 0.005 |
| zag05 | 5′reg | 110 | T | C | 92 | 0.549 | 0.451 |
| zag04 | 5′reg | 16 | T | A | 92 | 0.745 | 0.255 |
| zag03 | 5′reg | 132 | T | C | 92 | 0.022 | 0.978 |
| zag07 | Intron1 | 1347 | T | C | 93 | 0.823 | 0.177 |
| zag08 | Intron1 | 65 | T | G | 93 | 0.005 | 0.995 |
| zag09 | Intron1 | 2 | G | A | 93 | 0.027 | 0.973 |
| zag13 | Intron1 | 1766 | G | A | 93 | 0.995 | 0.005 |
| zag12 | Intron1 | 485 | T | C | 91 | 0.973 | 0.027 |
| zag10 | Intron1 | 355 | T | G | 91 | 0.709 | 0.291 |
| zag14 | exon2 | 1192 | T | C | 89 | 0.461 | 0.539 |
| zag23 | Intron2 | 169 | G | A | 91 | 0.456 | 0.544 |
| zag22 | Intron2 | 164 | T | C | 91 | 0.033 | 0.967 |
| zag18 | Intron2 | 308 | C | A | 92 | 0.75 | 0.25 |
| zag19 | Intron2 | 17 | G | A | 93 | 0.753 | 0.247 |
| zag20 | Intron2 | 235 | T | C | 93 | 0.968 | 0.032 |
| zag21 | Intron2 | 5 | T | C | 93 | 0.962 | 0.038 |
| zag17 | Intron2 | 588 | G | A | 92 | 0.527 | 0.473 |
| zag del | Intron2 | 30 | wt | del | 91 | 0.527 | 0.473 |
| zag16 | Intron2 | 423 | G | A | 90 | 0.533 | 0.467 |
| zag15 | Intron2 | 1048 | T | C | 91 | 0.478 | 0.522 |
| zag24 | Intron3 | 653 | C | A | 92 | 0.005 | 0.995 |
| zag25 | Intron3 | 237 | G | A | 93 | 0.995 | 0.005 |
| zag26 | Intron3 | 581 | T | G | 92 | 0.005 | 0.995 |
| zag35 | 3′reg | 975 | T | C | 93 | 0.478 | 0.522 |
| zag34 | 3′reg | 390 | T | G | 93 | 0.022 | 0.978 |

¹distance between the current SNP and the previous one
²number of DNA samples with sequencing data
wt: wild type sequence
del: sequence in which positions 8077-8083 are deleted

TABLE 3
Raw haplotype frequency table

| | | | | | | | | | | | | | |
|---|---|---|---|---|---|---|---|---|---|---|---|---|---|
| Marker | Alleles | H1 | H2 | H3 | H4 | H5 | H6 | H7 | H8 | H9 | H10 | H11 | H12 |

| | | | | | | | | | | | | | |
|---|---|---|---|---|---|---|---|---|---|---|---|---|---|
| zag05 | T/C | T | C | C | T | T | C | T | C | T | T | T | C |
| zag04 | T/A | T | T | T | A | A | T | T | A | A | T | A | T |
| zag07 | T/C | T | T | C | T | T | C | T | T | T | C | T | T |
| zag10 | T/G | G | T | T | T | T | T | T | T | T | G | T | T |
| zag14 | T/C | C | T | T | C | C | T | C | C | C | C | C | T |
| zag23 | GIA | A | G | G | A | A | G | A | A | A | A | C | C |
| zag18 | C/A | C | C | C | A | A | C | A | A | C | C | C | C |
| zag19 | C/A | G | G | C | A | A | G | A | A | G | G | G | C |
| zag17 | G/A | A | G | G | A | G | A | A | A | C | A | C | G |
| zag_del | wt/del | del | Wt | Wt | del | Wt | del | del | del | Wt | del | Wt | Wt |
| zag16 | C/A | A | G | G | A | C | A | A | A | G | A | G | G |
| zag15 | T/C | T | C | C | T | C | T | T | T | C | T | C | T |
| zag35 | T/C | T | C | C | T | C | T | T | T | C | T | C | C |

| | | | | | | | | | | | | |
|---|---|---|---|---|---|---|---|---|---|---|---|---|
| Haplotype F1 | 0.285 | 0.274 | 0.161 | 0.161 | 0.07 | 0.006 | 0.006 | 0.006 | 0.006 | 0.005 | 0.005 | 0.005 |

| | | | | | |
|---|---|---|---|---|---|
| Marker | Alleles | H13 | H14 | H15 | H16 |

| | | | | | |
|---|---|---|---|---|---|
| zag05 | T/C | T | T | T | T |
| zag04 | T/A | A | A | A | A |
| zag07 | T/C | T | T | C | C |
| zag10 | T/G | T | T | T | T |
| zag14 | T/C | T | T | C | C |
| zag23 | GIA | A | G | A | A |
| zag18 | C/A | A | A | A | C |
| zag19 | C/A | A | A | G | G |
| zag17 | G/A | A | A | A | A |
| zag_del | wt/del | del | del | del | del |
| zag16 | C/A | A | A | A | A |
| zag15 | T/C | T | T | T | T |
| zag35 | T/C | T | T | T | T |

| | | | | | |
|---|---|---|---|---|---|
| Haplotype F1 | 0.285 | 0.003 | 0.003 | 0.003 | 0.003 |

F is the Frequency. A test of Hardy-Weinberg (H-W) equilibrium was performed for each marker separately. No significant departure from H-W equilibrium was found at the 5% type I error. Haplotype frequency estimation conditions were met (Zhao et al., 2003).

The haplotypic characteristic of AZGP1 is commonly observed in other human genes in Caucasians: a set of few common haplotypes (here 5), and a series of rare haplotypes.

The alleles of the markers identified as associated with obesity (zag15, zag17 and zag35), were present at the heterozygote state in the L3 and L21 lean subjects (see table 4). The presence of those alleles in the two subjects with the lowest AZGP1 gene expression level provide some evidence of the importance of AZGP1 in the obesity status. This observation is reinforced by the genomic study performed which shows clearly that the L3 and L21 subjects are close to obese subjects when looking at their entire gene expression profile.

TABLE 4

Characteristics of markers identified in AZGP1
(associated with obesity) and AZGP1 mRNA expression
levels in the lean and obese patients (see EP 1548445).

| | | | | | | |
|---|---|---|---|---|---|---|
| | | | | | | AZGP1 mRNA |
| Subject | zag17 | zag_del | zag16 | zag15 | zag35 | expression levels |

| | | | | | | |
|---|---|---|---|---|---|---|
| L3 | AG | del/wt | AG | TC | TC | 54 |
| L7 | GG | wt/wt | GG | CC | CC | 257 |
| L8 | GG | wt/wt | GG | CC | CC | 223 |
| L10 | GG | wt/wt | GG | CC | CC | 115 |
| L11 | GG | wt/wt | GG | CC | CC | 69 |
| L17 | GG | wt/wt | GG | CC | CC | 98 |
| L21 | AG | del/wt | AG | TC | TC | 41 |
| O2 | AG | del/wt | AG | TC | TC | 18 |
| O9 | AG | del/wt | AG | TC | TC | 10 |
| O13 | AG | del/wt | AG | TC | TC | 8 |
| O14 | AA | del/del | AA | TT | TT | 13 |
| O15 | AA | del/del | AA | TT | TT | 12 |
| O16 | AG | del/wt | AG | TC | TC | 9 |
| O18 | AA | del/del | AA | TT | TT | 4 |
| O19 | GG | wt/wt | GG | CC | CC | 14 |
| 20 | GG | wt/wt | GG | CC | CC | 6 |

The p-values obtained from each Fisher's tests are presented in Table 5.

TABLE 5
Association results between each SNP and the obesity status

| | p-value | p-value |
|---|---|---|
| | (dominant | (recessive |
| SNP | coding) | coding) |
| zag04 | 1 | 0.476 |
| zag05 | 0.361 | 0.361 |
| zag07 | 0.198 | 0.214 |
| zag10 | 0.08 | 0.476 |
| zag14 | 0.361 | 0.361 |
| zag15 | 0.311 | 0.03 |
| zag17** | 0.03 | 0.311 |
| zag18 | 1 | —* |
| zag19 | 1 | 1 |
| zag23 | 0.361 | 0.361 |
| zag35 | 0.311 | 0.03 |

*uninformative coding, as all 21 individuals were in the same category.
**zag16 and the intronic deletion (zag_del) are not displayed in the table as they are completely redundant with zag17 (see Table 3).

Three markers were significant: zag15, zag17 (which represents zag16 and zag_del) and zag35.

Thus, the cluster of markers zag15, zag16, zag17, zag_del and zag35 from AZGP1 is associated with the obese status in samples from the Oestensson cohort (EP 1548445). As these five markers are strongly correlated (see Table 3), it is consistent to see that they provide the same strength of evidence.

Therefore, the present invention provides an isolated nucleic acid comprising SEQ ID No. 2, or a fragment thereof including position 8047, 8077-8083, 8500, 9556 or 12002, except for a single polymorphic change at one of the positions as shown below:

zag15 at position 9556, wherein the T at this position is replaced by a C

zag16 at position 8500, wherein the A at this position is replaced by a G

zag17 at position 8047, wherein the A in this position is replaced by a G

zag_del at position 8077-8083, wherein the nucleic acids in these positions are deleted

zag35 at position 12002, wherein the T in this position is replaced by a C.

These polymorphisms are the basis for a method of determining the predisposition of an individual to obesity, comprising the steps of: a) isolating a nucleic acid from a sample that has been removed from the patient and b) detecting the nucleotide present at one or more polymorphic sites within Seq ID No. 2 as listed hereinbefore, wherein the presence of the nucleotide specified at the polymorphic site as listed hereinbefore is indicative of a propensity of a patient to obesity.

The polymorphisms described hereinbefore define several haplotypes in the AZGP1 to gene that are associated with obesity. Therefore, the present invention also provides an isolated nucleic acid molecule selected from the group consisting of haplotypes 1, wherein each of haplotypes 1-3 comprises SEQ ID No. 2 with the exception that the nucleotides specified in the table 6 below for each haplotype are present at the corresponding position in Seq ID No. 2:

TABLE 6
Haplotypes for markers of interest

| Position | Haplotype 1 | Haplotype 2 | Haplotype 3 |
|---|---|---|---|
| 8047 | A | G | G |
| 8077-8083 | del | wt | wt |
| 8500 | A | G | G |
| 9556 | T | C | T |
| 12002 | T | C | C |

As used herein, the term “del” relates to a sequence derived from Seq ID No. 1, wherein the nucleic acids from 8077 to 8083 in Seq ID No. 2 are deleted from the corresponding position in Seq ID No. 1. The term “wt” relates to a sequence derived from ID No. 2 wherein the nucleic acids from positions 8077 to 8083 are present.

Furthermore, a method for haplotyping the AZGP1 gene in an individual is provided comprising the steps of a) isolating a nucleic acid from a sample that has been removed from the individual; b) determining the presence of the nucleotides present at positions 8047, 8077-8083, 8500, 9556 and 12002 of the individual's copy of gene AZGP1, wherein the position numbers are determined by comparison to SEQ ID No. 2; c) assigning the individual a particular haplotype by comparison of the nucleotides present at said positions to the nucleotides recited in the haplotypes of the table 6 set forth hereinbefore. Preferably, the presence of at least one of the haplotypes set forth in the table 6 is indicative of the propensity of the individual to obesity.

The expression levels of ˜5000 different genes in fat biopsies taken from 7 lean and 9 obese were measured by high-density oligonucleotides microarray. This constituted their gene expression profile. A correspondence analysis (Benzecri J P. L'analyse des données. Dunod, Paris; 1979; Greenacre M. Theory and application of Correspondence Analysis. 1984; Academic Press, London; Fellenberg K, Hauser N, Brors B, Neutzner A, Hoheisel J D, and Vingron M. Correspondence analysis applied to microarray data. PNAS 1998:10781-86) was then performed on these gene expression levels. Each data point in FIG. 2 represents a projection of the entire gene expression profile of one subject in a three-dimensional space, as determined by correspondence analysis. The distance between subjects reflects the distance between their entire gene expression profiles.

All obese subjects—but O16 patient—are located on the right side of the F3 axis while the lean subjects are on the left side of this same axis, but four lean subjects—L3, L11, L17 and L21—who appear among the obese subjects.

From the statistical work performed, many differentially expressed genes were found when obese subjects were compared to lean ones. The AZGP1 gene, which is among these differentially expressed genes, appears down-regulated in obese subjects compared to lean subjects (see graph 2, fold change=−11.5 with a P-value<5%). The lean subjects having the lowest AZGP1 gene expression level (L3 and L21) are also the ones who appear close to the obese subjects in FIG. 2. The clinical parameters of those same lean subjects indicate that their percentage of truncal fat is higher than in the lean subjects who exhibit a high level of AZGP1 mRNA. L21 has also a very low value of energy expenditure, compared to the values observed for the other lean subjects.

### SHORT DESCRIPTION OF THE FIGURES

FIG. 1: Markers of interest mapped on the genomic sequence used for SNP discovery in AZGP1. The following sequence is derived from the EMBL accession number ac004977. Markers of interest are highlighted (SNPs and deletion described in the statistical analysis). In this sequence, the deletion of zag_del is present.

FIG. 2: Correspondence analysis performed on the entire gene expression profiles of 7 lean and 9 obese subjects, measured with high-density oligonucleotide microarray. Each data point corresponds to the entire gene expression profile of one subject. Lean subjects are depicted by black squares and obese subjects by grey squares. The analysis was performed using the statistical package XlStat 6.0 (Addinsoft; New York, N.Y.).

FIG. 3: AZGP1 expression profile measured with high-density oligonucleotide microarray (see values in table 4).

### EXAMPLES

### Example 1

### DNA Samples

DNA samples used for SNP discovery were from two different origins:
Most of them were purchased directly as DNA samples from the Coriell Institute for Medical Research.

Twenty one of them were prepared at RCMG from whole blood provided by Professor Claes Oestenson (see EP 1548445). All patients were non diabetic at the time when samples were taken. DNA was extracted from 200 μl of the whole blood using a silica gel-based extraction method (MagNA Pure LC DNA Isolation KIT I, Roche Molecular Biochemicals).

### Example 2

### SNP Discovery

The mRNA sequence of AZGP1 (NCBI accession number NM_{—}001185) was mapped on the genomic sequence (EMBL accession number ac004977, LocusLink 563) to identify the genomic organization of AZGP1 (exons and exons/intron boundaries). Primers were designed to amplify DNA fragments that would cover the whole gene sequence and additionally 1.5 kb upstream AZGP1 start codon (ATG) and 1 kb downstream AZGP1 stop codon (TAG) (Table 7). These fragments are overlapping each other. Fragments were amplified by PCR using DNA sample from several individuals as a template. The amplification conditions were as following, in a final volume of 20 μl:

4 ng DNA

Buffer 1× (see Table 8 for details)

50 μM of each dATP, dCTP, dGTP and dTTP

0.4 μM of each primer

0.4 u of polymerase (see Table 8 for details)

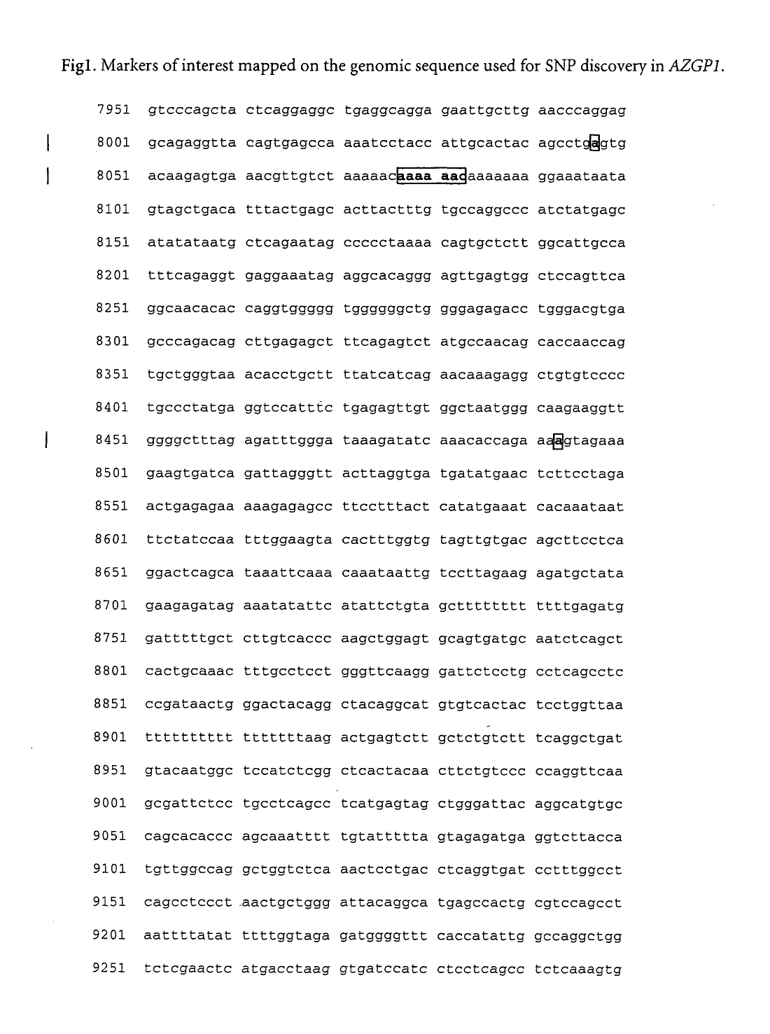

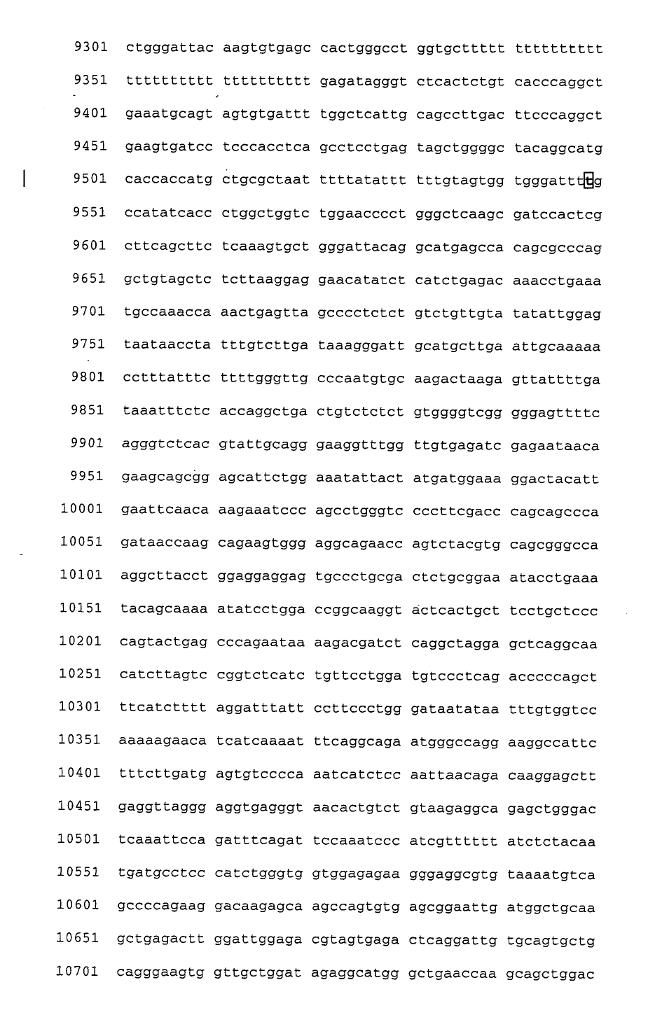

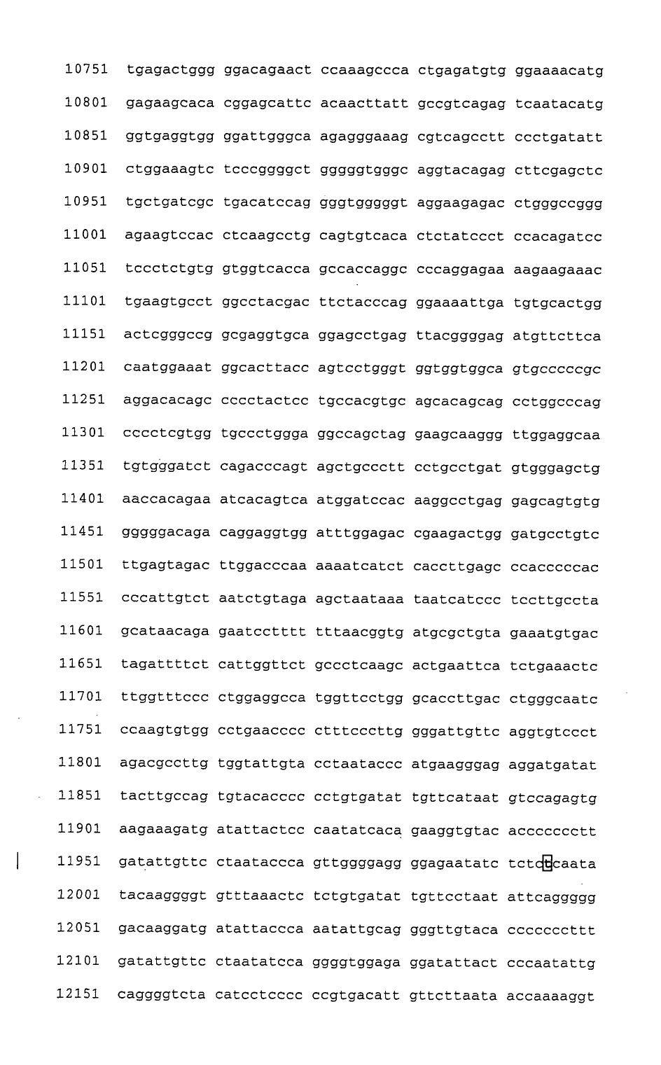

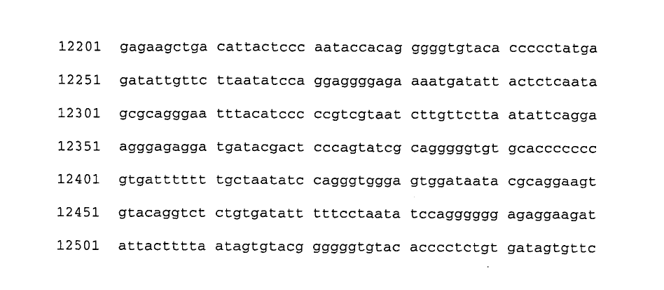

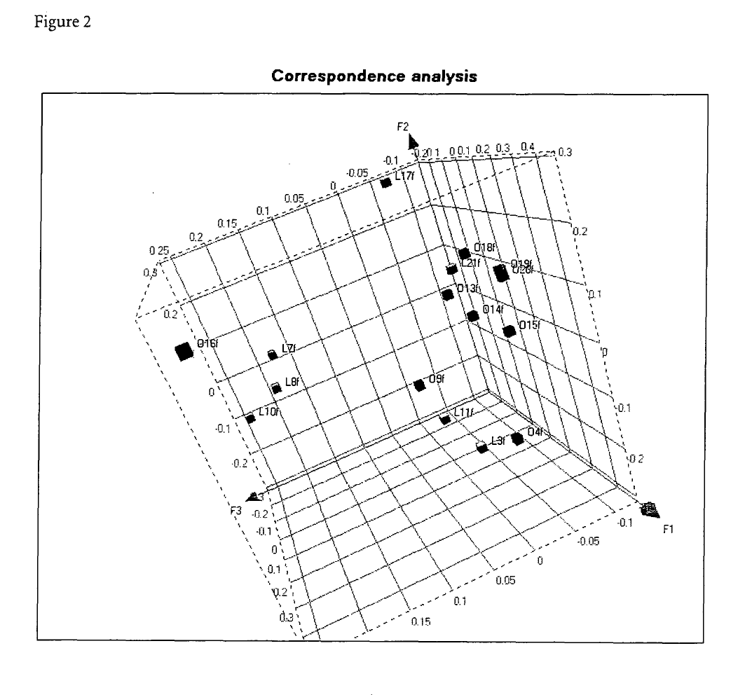

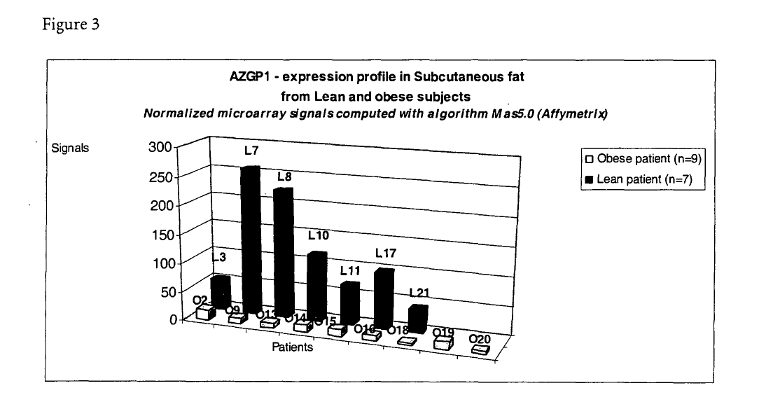

Amplification reactions were prepared in 96-well amplification plates with an aliquoting robot (Tecan biorobot). Parameters for procedures performed by the robot were set to minimize the possibility of cross-contamination. The thermal cycling conditions were as following: 15 minutes at 95° C. for DNA polymerase activation, 35 cycles of the following steps: denaturation at 94° C. for 1 min, hybridization at the annealing temperature (Table 8) for 30 s and extension at 72° C. for 1 min, and a final extension step at 72° C. for 5 min. The amplification reactions were run on an MJ Research PTC-200 DNA Engine. After PCR amplification, fragments were purified using 384 Cleanup Millipore plates on a Tecan biorobot. Double strand DNA sequencing of all fragments was performed using ABI Big Dye terminator chemistry according to the manufacturer's instructions. Primers used for sequencing were the same as the ones used for fragment amplification. Sequencing reactions were performed on an MJ Research PTC-200 DNA Engine and run on an ABI 3730 sequencer. After sequencing, the polymorphism analyses were done using Polyphred software (licensed from University of Washington). Table 3 is listing all markers identified in AZGP1. Position of these markers on AZGP1 genomic sequence is also highlighted in FIG. 1.

TABLE 7
Primers used to amplify and sequence AZGP1.

| | | | | |
|---|---|---|---|---|
| | Primer | Begin | End | |
| Primer name | No | position1 | position¹ | Sequence |

| | | | | |
|---|---|---|---|---|
| AZGP1-5Reg-F | 3 | 1941 | 1961 | GTCCAAAAACACACAAATGCC |
| AZGP1-5Reg-R | 4 | 2441 | 2422 | TTCCTCACCTCCTTCCAGTC |
| AZGP1-5Regc-F | 5 | 694 | 713 | TCCAACCAACAGCATGTAAG |
| AZGP1-5Regc-R | 6 | 1539 | 1520 | CCCTCCGAATACAAAGCAAC |
| AZGP1-ex01-F | 7 | 2290 | 2309 | AGAACCCTCCAAGCAGACAC |
| AZGP1-ex01-R | 8 | 3107 | 3084 | GGCACAGAATCAGATTAACATTCC |
| AZGP1-ex02-F | 9 | 5815 | 5835 | TTCTAACGCATGTCAGATTCC |
| AZGP1-ex02-R | 10 | 6678 | 6658 | CTATTTCCATCCTGCTGATCC |
| AZGP1-ex03-F | 11 | 9685 | 9704 | TGAGACAAACCTGAAATGCC |
| AZGP1-ex03-R | 12 | 10191 | 10173 | AAGCAGTGAGTACCTTGCC |
| AZGP1-ex04-F | 13 | 10614 | 10633 | AAGAGCAAGCCAGTGTGAGC |
| AZGP1-ex04-R | 14 | 11474 | 11453 | AAATCCACCTCCTGTCTGTCCC |
| AZGP1-in03-F | 15 | 10042 | 10060 | AGCAGCCCAGATAACCAAG |
| AZGP1-in03-R | 16 | 10832 | 10812 | GCAATAAGTTGTGAATGCTCC |
| AZGP1-in02-F | 17 | 8970 | 8989 | GCTCACTACAACTTCTGTCC |
| AZGP1-in02-R | 18 | 9822 | 9801 | GGCAACCCAAAAGAAATAAAGG |
| AZGP1-in02b-F | 19 | 8245 | 8263 | AGTTCAGGCAACACACCAG |
| AZGP1-in02b-R | 20 | 9108 | 9089 | GGCCAACATGGTAAGACCTC |
| AZGP1-in02c-F | 21 | 7568 | 7587 | GGCAAGAAAGAGATAGGCAG |
| AZGP1-in02c-R | 22 | 8432 | 8412 | CCACAACTCTCAGAAATGGAC |
| AZGP1-in02d-F | 23 | 6945 | 6964 | AGCCACTCTCAAAGTCACTC |
| AZgP1-in02d-R | 24 | 7798 | 7777 | AGCCCTGCCTTCTATTATTTTC |
| AZGP1-in02e-F | 25 | 6474 | 6493 | ACAGGTGGAAGGAATGGAGG |
| AZGP1-in02e-R | 26 | 7156 | 7137 | TAGGTGATGGAGCTGCAAGG |
| AZGP1-in01-F | 27 | 5094 | 5115 | CTTACCCTGTGCTAATTCAGTG |
| AZGP1-in01-R | 28 | 5967 | 5947 | GTCCCTTTTGTTTCTCATCCC |
| AZGP1-in01b-F | 29 | 4767 | 4786 | TACCCATTAACCACCCTCCC |
| AZGP1-in01b-R | 30 | 5547 | 5527 | GCTTGGATGACAGAGTGAGAC |
| AZGP1-in01c-F | 31 | 4087 | 4106 | GGATTCTTGTTCTGTCACCC |
| AZGP1-in01c-R | 32 | 4916 | 4895 | CTTGCTCCTGAGTGTCTAAATG |
| AZGP1-in01d-F | 33 | 3464 | 3483 | GGATGAAGCCCACCACTATG |
| AZGP1-in01d-R | 34 | 4272 | 4253 | GGTCAAGAGGTCAAGACCAG |
| AZGP1-in01e-F | 35 | 2837 | 2856 | CCCAAATCCCACACTCAGAC |
| AZGP1-in01e-R | 36 | 3652 | 3633 | AGCTTGAAGGGATGGATACC |
| AZGP1-3REG-F | 37 | 11199 | 11219 | CACAATGGAAATGGCACTTAC |
| AZGP1-3REG-R | 38 | 11990 | 11971 | GATATTCTCCCCTCCCCAAC |
| AZGP1-3REGb-F | 39 | 11780 | 11763 | TGAACCCCCTTTCCCTTG |
| AZGP1-3REGb-R | 40 | 12500 | 12483 | ATCTTCCTCTCCCCCCTG |

¹Position in SEQ ID No. 1

TABLE 8
Amplification conditions for all fragments

| | Primer | Annealing | | |
|---|---|---|---|---|
| Fragment | Numbers | temperature | Buffer | Polymerase |
| 5Reg | 3/4 | 58 | FastStart buffer | AmpliTaqGold |
| | | | (Roche) | |
| 5Regc | 5/6 | 60 | FastStart buffer | AmpliTaqGold |
| | | | (Roche) | |
| ex01 | 7/8 | 62 | FastStart buffer | AmpliTaqGold |
| | | | (Roche) | |
| ex02 | 9/10 | 62 | FastStart buffer | AmpliTaqGold |
| | | | (Roche) | |
| ex03 | 11/12 | 58 | FastStart buffer | AmpliTaqGold |
| | | | (Roche) | |
| ex04 | 13/14 | 62 | FastStart buffer | AmpliTaqGold |
| | | | (Roche) | |
| in03 | 15/16 | 60 | FastStart buffer | AmpliTaqGold |
| | | | (Roche) | |
| in02 | 17/18 | 60 | FastStart buffer | AmpliTaqGold |
| | | | (Roche) | |
| in02b | 19/20 | 62 | Roche buffer² | AmpliTaqGold |
| in02c | 21/22 | 60 | FastStart buffer | AmpliTaqGold |
| | | | (Roche) | |
| in02d | 23/24 | 60 | FastStart buffer | AmpliTaqGold |
| | | | (Roche) | |
| in02e | 25/26 | 62 | FastStart buffer | AmpliTaqGold |
| | | | (Roche) | |
| in01 | 27/28 | 62 | FastStart buffer | AmpliTaqGold |
| | | | (Roche) | |
| in01b | 29/30 | 62 | FastStart buffer | AmpliTaqGold |
| | | | (Roche) | |
| in01c | 31/32 | 62 | FastStart buffer | AmpliTaqGold |
| | | | (Roche) | |
| in01d | 33/34 | 62 | Buffer D | Taq |
| | | | (Invitrogen)³ | |
| in01e | 35/36 | 60 | FastStart buffer | AmpliTaqGold |
| | | | (Roche) | |
| 3Reg | 37/38 | 62 | FastStart buffer | AmpliTaqGold |
| | | | (Roche) | |
| 3Regb | 39/40 | 58 | FastStart buffer | AmpliTaqGold |
| | | | (Roche) | |

¹FastStart buffer 1x: 50 mM Tris-HCl, 10 mM KCl, 5 mM (NH₄)₂SO₄, 2 mM MgCl₂, pH 8.3 25° C.
²Roche buffer 1x: 10 mM Tris-HCl, 50 mM KCl, 1.5 mM MgCl₂, pH 8.3 25° C.
³Buffer D 1x: 30 mM Tris-HCl, 7.5 mM (NH₄)₂SO₄, 3.5 mM MgCl₂, pH 8.5 25° C.

### Example 3

### Haplotype Frequency Estimation Method

Haplotype frequencies were estimated using an E-M algorithm as implemented in Genecounting (Zhao J H, Lissarrague S, Essioux L, Sham PC. GENECOUNTING: haplotype analysis with missing genotypes. Bioinformatics. 2002 Dec., 18(12):1694-5). This program takes into account individuals with untyped sites, and is thus providing more accurate estimations.

### Example 4

### Analysis of the Deletion Findings in 10 Obese/11 Lean

The genomic sequence of AZGP1 was sequenced in 10 obese patients and 11 lean samples from Professor Oestenson's cohort (EP 1548445). All frequent SNPs from Table 3 were present. Association tests between the obese status and the genotypes were carried in the 11 non-redundant frequent SNPs: zag05, zag04, zag07, zag10, zag14, zag23, zag18, zag19, zag17/zag16/zag del, zag15 and zag35.

Compared to previous analyses zag18 from zag19 could be separated. They were thus treated as two non redundant SNPs.
Two coding schemes were applied:
A dominant coding where the heterozygotes and the homozygotes for the rarer allele are pooled in one category.
A recessive coding where the heterozygote and the homozygotes for the most common allele are pooled in one category.
   Under each coding scheme, each genotypic variable is a binary variable. For each variable created an exact 2×2 fisher test was performed. The significance threshold taken was 0.05.

### Example 5

### AZGP1 mRNA Profiling in Lean and Obese Subjects

Subcutaneous fat biopsies were obtained from the twenty one subjects coming from the cohort described in EP 1548445. For five subjects (L1, L5, L12, O2 and O6), it was not possible to perform microarrays with the corresponding biopsies.

A gene expression study was performed using high-density oligonucleotide microarray gene technology provided by Affymetrix (Affymetrix GeneChip® Technology; Affymetrix, Inc.; Santa Clara, Calif.) on the remaining sixteen samples.

### Example 5.1

### RNA Preparation

Total RNA from 500 mg subcutaneous fat tissue was isolated using the TriZol reagent (Life Technologies) and the Fast RNA green (BIO101) kit according to the manufacturer's protocols. Total RNA was purified from contaminating DNA using the RNeasy kit (Qiagen).

### Example 5.2

### Gene Expression Profiling by High-Density Oligonucleotide Microarray

Syntheses of first and second strand cDNA were performed using the SuperScript Choice Gene Chip Kit (Life Technologies) and reagents from Gibco. Double stranded cDNA, containing an incorporated T7 RNA polymerase binding site, was purified by extraction with a mix of phenol: chloroform: isoamylalcohol (v/v/v. 25/24/1, Life Technologies). The organic and aqueous phases were separated by Phase Lock Gel (Eppendorf) and double stranded cDNA was recovered by precipitation according to the manufacturer's protocol and then resuspended in water. Double stranded cDNA was converted to biotin-labeled cRNA by in vitro transcription (IVT) using a T7 kit (Ambion) and biotin-containing ribonucleotides (Enzo-LOXO GmbH). The IVT-material was purified from unincorporated ribonucleotides using RNeasy spin columns (Qiagen). Following cleanup, single stranded biotin-labeled cRNA was chemically hydrolyzed to smaller fragments in 500 mM calcium acetate, 150 mM magnesium acetate, pH 8.1 for 35 min at 95° C. The reaction was terminated by chilling samples on ice.

One U95-A Affymetrix GeneChip Microarray was hybridized per sample. Each microarray contains 12559 probe sets representing ˜10,000 genes. All washing, hybridization, detection, and signal amplification steps were performed using a GeneChip Fluidics Station (Affymetrix Inc.; Santa Clara, Calif.). Fluorescence intensity data was collected from the hybridized GeneArrays using a GeneArray scanner (Affymetrix Inc.; Santa Clara, Calif.). The raw files containing the fluorescence intensity information were transformed into data files using the MAS 5.0 algorithm (component of GCOS 1.0 software). Only 45% of the genes mapped on the microarray were used in the analysis as the rest of them were called absent by the MAS 5.0 algorithm. Differentially expressed genes were identified using the Roche Affymetrix Chip Experiment Analysis (RACE-A) software.

### Example 6

### Genotyping of zag14, zag15 and zag16

### Example 6.1

### Cohort Description

86 impaired glucose tolerant (IGT) obese male patients and 290 normal glucose tolerant (NGT) male controle subjects, with normal BMI (BMI<25 Kg/m²), were studied. All were Swedish male patients selected from the Stockholm Diabetes Prevention Program. IGT obese subjects had normal birth weight, normal BMI (<25 Kg/m²), and normal plasma glucose levels 2 hours after oral glucose tolerance tests. Concentrations of plasma glucose, plasma insulin, and other clinical characteristics were measured as described in Gu et al., (Single nucleotide polymorphisms in the proximal promoter region of the adiponectin (APM1) gene are associated with type 2 diabetes in Swedish caucasians, Diabetes 53 Suppl 1: 31-5, 2004). Informed consent was obtained from all subjects, and the study was approved by the local ethics committees. Genomic DNA was extracted from peripheral blood using a Puregene DNA purification kit (Gentra) (Gu et al., supra).

### Example 6.2

### PCR-Dynamic Allele-Specific Hybridization (DASH) Assay Design and Genotyping

A high throughput SNP (Single Nucleotide Polymorphism) scoring technique called dynamic allele-specific hybridization (DASH) (Howell, et al., Dynamic allele-specific hybridisation: a new method for scoring single nucleotide polymorphisms, Nat Biotech 17: 87-88, 1999) was used for SNP genotyping. PCR-DASH assay design and SNP genotyping were performed as described previously (Prince, et al., Robust and accurate single nucleotide polymorphism genotyping by dynamic allele-specific hybridization (DASH): design criteria and assay validation, Genome Res 11: 152-162, 2001).

### Example 6.3

### Statistical Analyses

The aim of the statistical analysis was to confirm the previous results: at the genetic polymorphism zag15, patients homozygotes TT and heterozygotes CT were at higher risk of being IGT obese when compared to patients homozygotes CC. A 2-by-2 contingency table was formed. The statistical test hypotheses were, using unilateral alternatives hypotheses:

Null hypothesis (H0): p1=p0
Alternative hypothesis (H1): p1>p0

The parameters p1 and p0 are proportions of patients carrying at least one copy of the T allele at zag15 among IGT obese patients and controls respectively. The statistical test for proportion comparison was based on the normality of the arcsinus-transformed proportions. Under the null hypothesis, the test follows a normal distribution N(0,1). An excat test of proportion was also added (Agresti, Categorical data analysis. New York: Wiley, pp. 59-66, 1990).

The test was performed at the type I error of 5%.

The odd ration (OR) of developing impaired glucose tolerance and obesity associated with the tested genetic characteristics at the SNP zag15 was computed. The corresponding 95% confidence intervals were computed using the free statistical software R.

The table below is showing the distribution of each genotype at zag15 between the two patients groups.

| | Obese IGT | Normal NGT | Total |
|---|---|---|---|
| TT | 28 | 67 | 95 |
| CT | 40 | 132 | 172 |
| CC | 18 | 87 | 105 |
| Total | 86 | 286 | 372 |

The proportion of TT and CT patients was 0.79 in the obese IGT group compared to 0.7 in the control group. Carrying at least one copy of the T allele increased the odds of being IGT obese by 1.65 (95% CI: [0.93; 2.94]. The null hypothesis of independence between the genetic model and the obese IGT status was rejected versus a higher proportion of TT/CT patients in the obese IGT group at the 5% level (z=1.77, p=0.04). Using the excat proportion test (Agresti, supra), the results were borderline significant (p=0.055).

With this extended cohort coming from the same ethnic origin and prevention study as described in Examples 1-5 the genetic association between zag15 and the obesity impaired glucose tolerance status was confirmed. In view of the complete genetic equivalence between the polymorphism zag15, zag16, zag_del, zag17 and zag35, the association is also holding true for all polymorphism in this cluster, namely zag16, zag17, zag_del and zag35.

1. An isolated nucleic acid comprising SEQ ID No. 2 except for a single polymorphic change at one of the positions as shown below:
zag15 at position 9556, wherein the T at this position is replaced by a C
zag16 at position 8500, wherein the A at this position is replaced by a G
zag17 at position 8047, wherein the A in this position is replaced by a G
zag_del at position 8077-8083, wherein the nucleic acids in these positions are deleted
zag35 at position 12002, wherein the T in this position is replaced by a C.

2. A method of deterring the predisposition of an individual to obesity, comprising the steps of:
a) isolating a nucleic acid from a sample that has been removed from the individual and
b) detecting the nucleotide present at one or more polymorphic sites within SEQ ID No. 2 as listed in claim 1, wherein the presence of the nucleotide specified at the polymorphic site of claim 1 is indicative of a propensity of an individual to obesity.

3. An isolated nucleic acid molecule selected from the group consisting of haplotypes 1-3, wherein each of haplotypes 1-3 comprises SEQ ID No. 2 with the exception that the nucleotides specified in the table below for each haplotype are present at the corresponding position in SEQ ID No. 2:

| Position | Haplotype1 | Haplotype 2 | Haplotype 3 |
|---|---|---|---|
| 8047 | A | G | G |
| 8077-8083 | del | wt | wt |
| 8500 | A | G | G |
| 9556 | T | C | T |
| 12002 | T | C | C |

4. A method for haplotyping the AZGP1 gene in an individual comprising the steps of:
a) isolating a nucleic acid from a sample that has been removed from the individual;
b) determining the presence of the nucleotides at positions 8047, 8077-8083, 8500, 9556 and 12002 of the individual's copy of gene AZGP1, wherein the position numbers are determined by comparison to SEQ ID No. 2, and;
c) assigning the individual a particular haplotype by comparison of the nucleotides present at said positions to the nucleotides recited in the haplotypes of the table set forth in claim 3.

5. (canceled)

6. The method of claim 4, wherein the presence of at least one of the haplotypes set forth in the table of claim 3 is indicative of the propensity of the individual to obesity.
